(19) United States
(12) Patent Application Publication
(10) Pub. No.: US 2012/0016420 A1
(43) Pub. Date: Jan. 19, 2012

(54) DEVICES, SYSTEMS, AND METHODS FOR INTER-TRANSVERSE PROCESS DYNAMIC STABILIZATION

(76) Inventor: FRED F. NARAGHI, San Francisco, CA (US)

(21) Appl. No.: 13/091,600

(22) Filed: Apr. 21, 2011

Related U.S. Application Data
(60) Provisional application No. 61/399,585, filed on Jul. 14, 2010.

(51) Int. Cl. A61B 17/70; A61B 17/88

(52) U.S. Cl. 606/250; 606/279

(57) ABSTRACT

Systems and methods are positioned between left and right transverse processes of adjacent vertebrae in a spine to provide dynamic inter-transverse process distraction and stabilization and indirect expansion of both anterior and posterior spaces of the spine. The systems and methods employ a left support component sized and configured to be mounted between the left transverse processes of the adjacent vertebrae and a right support component and configured to be mounted between the right transverse processes of the adjacent vertebrae. The left and right support components exert a dynamic separation force between the left and right transverse processes generally longitudinally along the spine to distract and stabilize space between the adjacent inferior and superior vertebrae and indirectly expand both anterior and posterior spaces of the spine. The systems and methods relieve pain associated with, e.g. disc herniation, disc degeneration, facet arthropathy, and spinal stenosis.

### RELATED APPLICATION

This application claims the benefit of U.S. Provisional Patent Application Ser. No. 61/399,585, filed 14 Jul. 2010, and entitled “Devices, Systems, and Methods for Inter-Transverse Process Dynamic Stabilization,” which is incorporated herein by reference.

### FIELD OF THE INVENTION

The invention generally relates to devices, systems and methods for treating conditions of the spine, and, in particular, systems and methods for distending the spine for treating, e.g., disc herniation and spinal stenosis.

### BACKGROUND OF THE INVENTION

The spine is made up of bones (vertebrae) cushioned by intervertebral discs. The intervertebral discs are responsible for the attachment of vertebral bodies to each other, providing flexibility and load-sharing for the spinal column. An intervertebral disc consists of a tough outer layer (annulus) and a soft inner layer (nucleus).

With aging, a disc can undergo significant changes in volume and shape as well as in biochemical composition and biomechanical properties. The disc can become herniated, which is sometimes colloquially called a “slipped” disc or a “ruptured” disc. Other terms that are closely related include disc protrusion, bulging disc, pinched nerve, sciatica, disc disease, disc degeneration, degenerative disc disease, and black disc.

When a herniated disk occurs, a small portion of the nucleus pushes out through a tear in the annulus into the spinal canal. The annulus tears usually at the back of the disc, which is right next to the nerves of the spine. The nucleus starts to shift into the torn area, which causes a bulge. The bulge applies pressure to the nerve. The bulge can irritate a nerve and result in pain, numbness or weakness in the back, as well as in a leg or an arm. Disc herniation can cause back pain and or leg pain via compression of nerve roots. Pain can also occur due to the nucleus material causing chemical irritation of adjacent neural pathways such as nerve roots, dura, and the posterior longitudinal ligament.

Disc herniation can occur in any disc in the spine, but the two most common forms are lumbar disc herniation and cervical disc herniation. The former is the most common, causing lower back pain (lumbago) and often leg pain (sciatica) as well.

The most common levels for a herniated disc are L4-5 and L5-S1. The onset of symptoms is characterized by a sharp, burning, stabbing pain radiating down the posterior or lateral aspect of the leg, to below the knee. Pain is generally superficial and localized, and is often associated with numbness or tingling. In more advanced cases, motor deficit, diminished reflexes or weakness may occur.

Spinal degeneration can also cause a medical condition called spinal stenosis, in which the spinal canal narrows and compresses the spinal cord and nerves. Spinal stenosis can be caused by spinal disc herniation, osteoporosis, a tumor, or a congenital condition. Spinal stenosis may affect the cervical, thoracic or lumbar spine. In some cases, it may be present in all three places in the same patient. Lumbar spinal stenosis results in low back pain as well as pain or abnormal sensations in the legs, thighs, feet or buttocks, or loss of bladder and bowel control.

Disc herniation and spinal stenosis can sometimes be treated without surgery, e.g., through the use of medications, steroid injections, rest or restricted activity, or physical therapy.

In cases when non-surgical treatments are not effective, surgical treatments can be performed. Lumbar spine surgeries are performed for treatment of disc herniation and spinal stenosis on several hundred thousand patients each year to alleviate back pain and leg pain. The traditional treatments for disc herniation and stenosis to alleviate pain symptoms have involved discectomy and decompression which usually involve removing bone from lamina partially (laminotomy), or completely (laminectomy), and removal of the herniated disc portion.

If there is a structural instability, typically the treatment would also include fusion. Fusion consists of application of bone or cage implants either in the interbody space or to the posterolateral portions of vertebral bodies with or without application of pedicle screw instrumentation, to stabilize the vertebrae and allow for fusion to occur to treat the instability.

Recent studies have questioned the effectiveness of discectomy, suggesting that discectomy may at best only offer a modest short-term benefit in patients with sciatica due to disc extrusion. According to these recent studies discectomy does not appear to provide a different outcome than non-surgical treatment. The advantage of discectomy is a more rapid resolution of the radicular leg symptoms. However, the disadvantages are numerous, including scar tissue, instability requiring further surgeries, nerve injury, accelerated disc degeneration. e.g., decompressive laminectomy, laminotomy, foraminotomy, cervical discectomy and fusion, cervical corpectomy, and laminoplasty.

### SUMMARY OF THE INVENTION

The invention provides devices, systems, and methods for treating back pain and leg pain by dynamic stabilization of transverse processes of adjacent vertebrae (either unilaterally left or right, or bilaterally left and right), optionally with facet joint fixation. More particularly, the invention provides devices, systems, and methods for distraction of the space between the transverse processes of spinal column (either unilaterally left or right, or bilaterally left and right), optionally with facet joint fixation.

The devices, systems, and methods that embody the technical features of the invention indirectly increase the area within the spinal canal, as well as the disc space, thereby providing an indirect front and back decompression of spinal canal and disc space. As a result, the devices, systems, and methods that embody the technical features of the invention can relieve the back and/or leg pain associated with impingement of the nerves due to disc herniation, disc degeneration, scoliosis, post-laminectomy syndrome, and spinal stenosis. The devices, systems, and methods that embody the technical features of the invention can provide a balanced distraction for both posterior and anterior portions of the spinal column, without causing kyphosis. The devices, systems, and methods that embody the technical features of the invention can optionally provide balanced facet joint fixation in tandem with balanced distraction for both posterior and anterior portions of the spinal column. The methods that embody the technical features of the invention also provide for the insertion of the devices and systems for distraction of the space between the transverse processes of spinal column to provide dynamic stabilization of transverse processes of adjacent vertebrae. The methods that embody the technical features of the invention can optionally also provide facet joint fixation in tandem with the distraction of the space between the transverse processes of spinal column, to provide dynamic stabilization of transverse processes and facet joints of adjacent vertebrae.

In one embodiment, the devices, systems, and methods are positioned between either left or right transverse processes of adjacent vertebrae in a spine to provide dynamic inter-transverse process distraction and stabilization and indirect expansion of anterior and/or posterior spaces of the spine. In this embodiment, the devices, systems, and methods include a support component sized and configured to be mounted between selected left or right transverse processes of the adjacent vertebrae.

The support component is manipulated to exert a dynamic separation force between the selected transverse processes generally longitudinally along the spine to distract and stabilize space between the adjacent inferior and superior vertebrae and indirectly expand anterior and posterior spaces of the spine, optionally with facet joint fixation. The devices, systems and methods can serve, e.g., to relieve pain associated with the spine, and/or treat pain, numbness, and/or weakness of a leg.

In one embodiment, the devices, systems, and methods are positioned between left and right transverse processes of adjacent vertebrae in a spine to provide dynamic inter-transverse process distraction and stabilization and indirect expansion of both anterior and posterior spaces of the spine. The devices, systems, and methods include a left support component sized and configured to be mounted between the left transverse processes of the adjacent vertebrae, and a right support component and configured to be mounted between the right transverse processes of the adjacent vertebrae. The left and right support components are manipulated to exert a dynamic separation force between the left and right transverse processes generally longitudinally along the spine to distract and stabilize space between the adjacent inferior and superior vertebrae and indirectly expand both anterior and posterior spaces of the spine, optionally with facet joint fixation. The devices, systems and methods can serve, e.g., to relieve pain associated with the spine, and/or treat pain, numbness, and/or weakness of a leg.

The devices, systems, and methods that incorporate the technical features of the invention can exert posterolateral biomechanical force in the inter-transverse process space, combining both anterior and posterior distraction and stabilization, and achieving posterolateral fusion, optionally with facet fixation. Prior techniques exert only posterior biomechanical force between the spinous processes. As such, none provides a posterolateral fusion. None exerts biomechanical force in the inter-transverse area of spine. The transverse process is located more anteriorly than the spinous process or even the facets. The transverse process is also closer to the discs in the anterior column of spine. Therefore, the net biomechanical effect of distracting the transverse processes is a combined anterior and posterior column distraction “indirectly”. The net biomechanical effect of distracting the transverse processes also avoids the potential for causing “kyphosis” or excessive forward bending/flexion angular deformity, which may possibly be associated with conventional spinous process distractors.

Other objects, advantages, and embodiments of the invention are set forth in part in the description which follows, and in part, will be obvious from this description, or may be learned from the practice of the invention.

### DESCRIPTION OF THE DRAWINGS

FIG. 1 is an anatomic view of a human spine, showing the different regions of vertebrae.

FIG. 2 is an anatomic ipsilateral view of the lower back region of the spine, showing the lumbar vertebrae L2 to L5, the sacral vertebrae S1 to S5, and the coccygeal vertebrae.

FIG. 3 is an anatomic posterior view of the lower back region of the spine, showing the lumbar vertebrae L2 to L5.

FIG. 4A is an anatomic top view of a vertebral body taken generally along line 4A-4A in FIG. 2, showing a normal intervertebral disc.

FIG. 4B is an anatomic top view of a vertebral body taken generally along line 4A-4A in FIG. 2, but showing a herniated intervertebral disc.

FIG. 5 is an exploded perspective view of a representative embodiment of a system that can be installed between transverse processes of adjacent superior and inferior vertebrae where a herniated disc exists, to provide dynamic stabilization of the vertebrae and relieves the pressure on the spinal cord and/or nerve roots caused by the herniated disc.

FIG. 6 is an assembled perspective view of the system shown in FIG. 5.

FIG. 7 is an anatomic perspective view of the lower back region of the spine, showing the system shown in FIGS. 5 and 6 after assembly and installation providing a balanced distraction for both posterior and anterior portions of the spinal column, without causing kyphosis.

FIGS. 8 to 10 are anatomic perspective views of the lower back region of the spine, showing the initial steps of installing the system shown in FIG. 7 including the initial placement of the support columns between the transverse processes.

FIG. 11 is a view of an instrument that can be used to adjust the support columns to provide a separating force between the adjacent vertebrae.

FIGS. 12 and 13 are anatomic perspective views of the lower back region of the spine, showing the manipulation of the instrument shown in FIG. 11 to provide a mechanical advantage while lengthening the support columns to provide a separating force between the adjacent vertebrae.

FIG. 14 is an anatomic perspective view of the lower back region of the spine, showing the support columns after being lengthened and locked to provide a desired distance between the adjacent vertebrae, thereby achieving the desired therapeutic benefit, e.g., to relieve pressure on the spinal cord and/or nerve roots occasioned by herniated condition of the disc.

FIG. 15 is a view of an instrument that can be used to provide a medial force to the support columns across the transverse processes to prior to assembly of the traverse brace components, which completes the installation of the system.

FIGS. 16 and 17 are anatomic perspective views of the lower back region of the spine, showing the manipulation of the instrument shown in FIG. 15 to provide a mechanical advantage while creating a medial force (in FIG. 16) that the brace components, when assembled to the system (in FIG. 17), maintain.

FIG. 18 is a perspective view of an alternative embodiment of a support column that the system shown in FIGS. 5, 6, and 7 can incorporate, the support column including fenestrations and sized to contain a bone graft material.

FIG. 19 is a perspective view of an alternative embodiment of a j-shaped rest carried by the support columns shown in FIG. 18, showing fenestrations that allow the bone graft material to extend from the support columns to the transverse processes, thereby providing a posterolateral fusion.

FIG. 20 is a perspective view of the fenestrated support column and j-shaped rests shown in FIGS. 18 and 19, and further showing the introduction of additional bone graft material into the support columns after assembly and installation of the system.

FIG. 21 is a perspective view of another representative embodiment of a system that can be installed between transverse processes of adjacent superior and inferior vertebrae where a herniated disc exists, to provide dynamic stabilization of the vertebrae as well as facet joint fixation, to relieve the pressure on the spinal cord and/or nerve roots caused by the herniated disc.

FIG. 22 is a medial elevation view of the transverse process hoist assembly and facet joint fixation bracket that form a part of the system shown in FIG. 21.

FIG. 23 is an anatomic perspective view of the lower back region of the spine, showing the system shown in FIGS. 21 and 22 after installation providing a balanced distraction and facet joint fixation for both left and right lateral portions of the spinal column.

FIG. 24 is an enlarged perspective view of the right side of the lower back region shown in FIG. 23, showing further details of the system distraction and facet joint fixation on the right lateral portion of the spinal column.

FIG. 25 is an anatomic perspective view of the lower back region of the spine, showing the system shown in FIGS. 21 and 22 after installation providing a balanced distraction and facet joint fixation for both left and right lateral portions of the spinal column, the system as shown in FIG. 25 further including superior and inferior brace components for enhanced stabilization.

FIGS. 26A and 26B are perspective views of an alternative embodiment of the hoist assembly and u-shaped rest carried by the hoist assembly, as shown in FIG. 21, and further showing fenestrations that allow bone graft material to extend from the hoist assembly and u-shaped rest to the transverse process, thereby providing a posterolateral fusion.

### DESCRIPTION OF PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims. While the present invention pertains to systems, devices, and surgical techniques applicable at virtually all spinal levels, the invention is well suited for achieving dynamic stabilization of transverse processes of adjacent lumbar vertebrae. It should be appreciated, however, the systems, device, and methods so described are not limited in their application to lumbar fusion and are applicable for use in treating different types of spinal problems.

### I. ANATOMICAL OVERVIEW

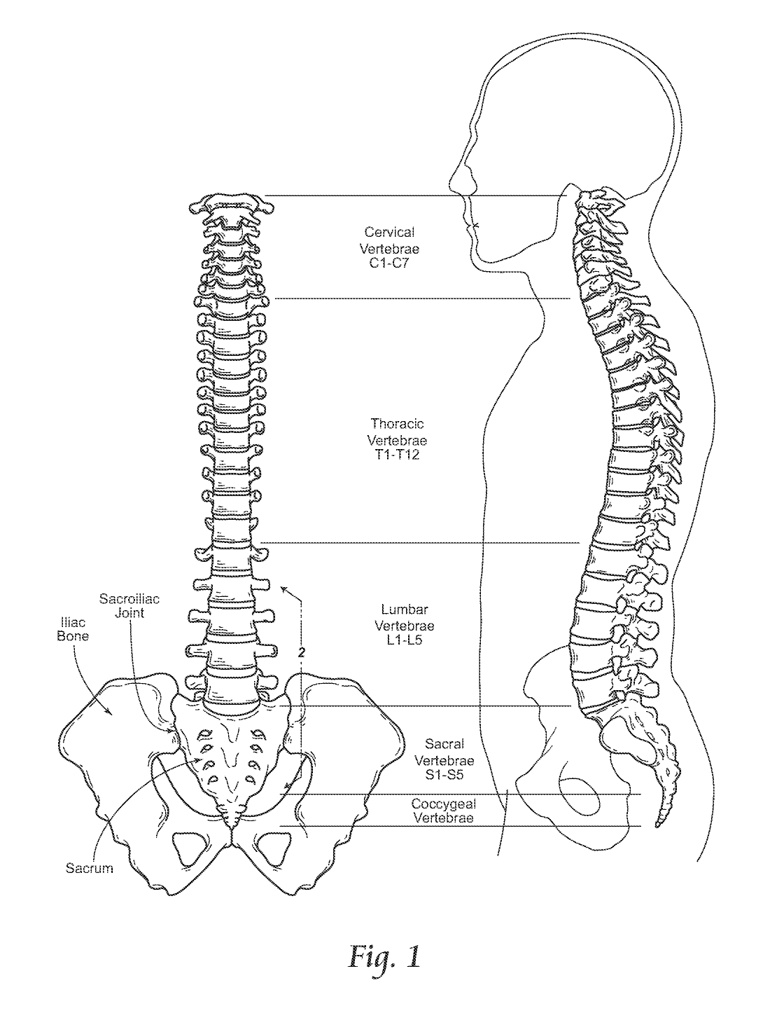

The spine (see FIG. 1) is a complex interconnecting network of nerves, joints, muscles, tendons and ligaments. The spine is made up of small bones, called vertebrae, which are named according to the region of the body they occupy. The vertebrae in the head and neck region are called the cervical vertebrae (designated C1 to C7). The vertebrae in the neck and upper back region are called the thoracic vertebrae (designated T1 to T12). The vertebrae in the lower back region are called the lumbar vertebrae (numbered L1 to L5). The vertebrae in the pelvic region are called the sacral vertebrae (numbered S1 to S5).

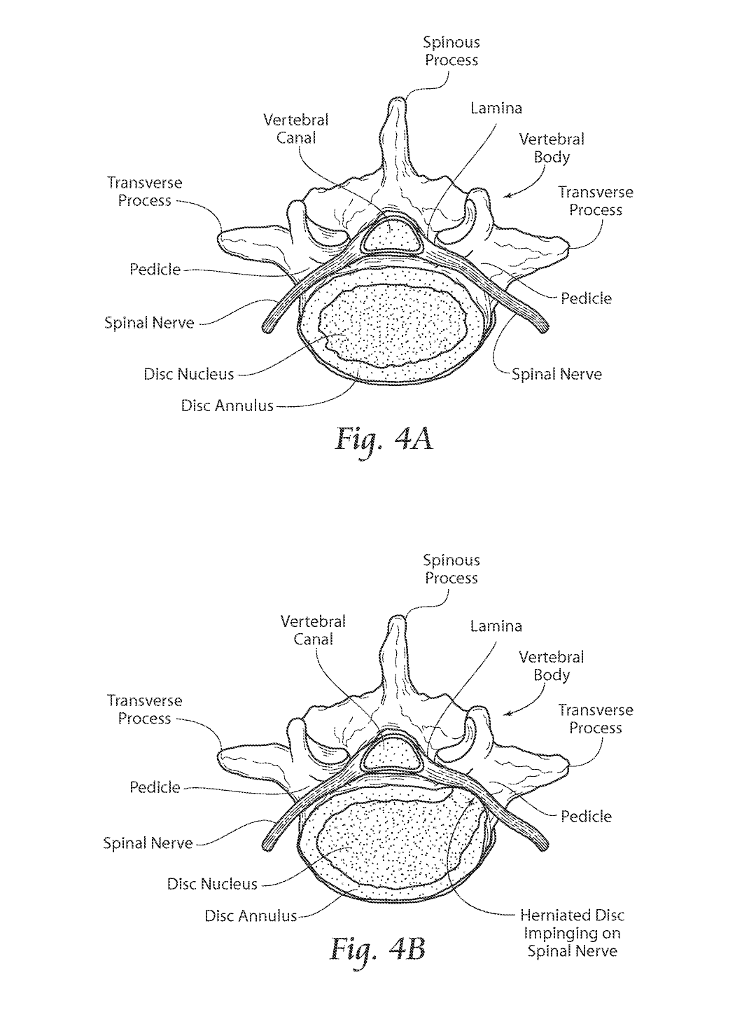

The vertebrae protect and support the spinal cord. They also bear the majority of the weight put upon the spine. As can be seen in FIG. 4A, vertebrae, like all bones, have an outer shell called cortical bone (the vertebral body) that is hard and strong. The inside is made of a soft, spongy type of bone, called cancellous bone. The bony plates or processes of the vertebrae that extend rearward and laterally from the vertebral body provide a bony protection for the spinal cord and emerging nerves.

The configuration of the vertebrae differ somewhat, but each (like vertebrae in general) includes a vertebral body (see FIG. 4A), which is the anterior, massive part of bone that gives strength to the vertebral column and supports body weight. The vertebral canal is posterior to the vertebral body and is formed by the right and left pedicles and lamina. The pedicles are short, stout processes that join the vertebral arch to the vertebral body. The pedicles project posteriorly to meet two broad flat plates of bone, called the lamina.

Other processes arise from the vertebral arch. For example, three processes—the spinous process and two transverse processes—project from the vertebral arch and afford attachments for back muscles, forming levers that help the muscles move the vertebrae.

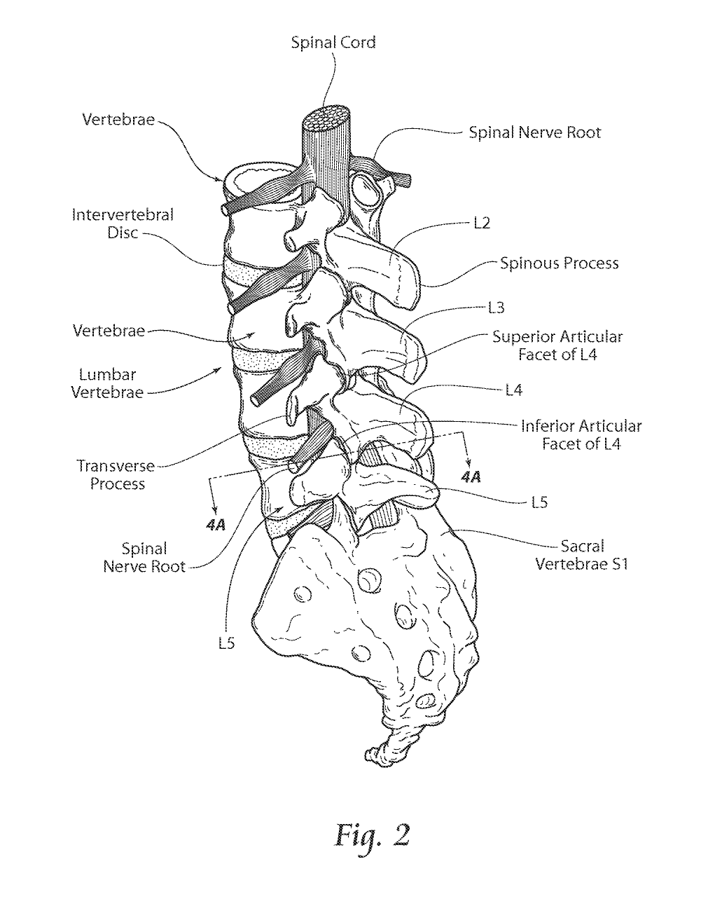

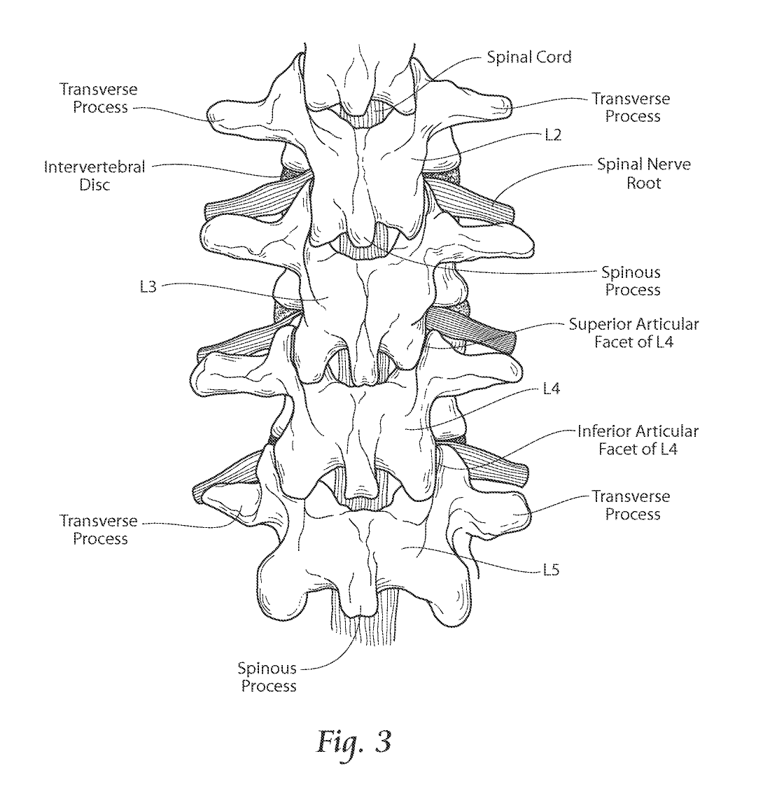

FIG. 2 shows the S1 sacral vertebra and the adjacent fourth and fifth lumbar vertebrae L4 and L5, respectively, in a lateral view (while in anatomic association). The sacral and lumbar vertebrae are in the lower back, also called the “small of the back.” FIG. 3 shows the fourth and fifth lumbar vertebrae L4 and L5 from a different, more posterior, perspective.

As previously described, between each vertebra is a soft, gel-like “cushion,” called an intervertebral disc (see FIG. 2). These flat, round cushions act like shock absorbers by helping absorb pressure and keep the bones from rubbing against each other. The intervertebral disc also binds adjacent vertebrae together. The intervertebral discs can bend and rotate a bit but do not slide.

FIG. 4A shows a vertebra with a normal intervertebral disc. FIG. 4B shows a vertebra with a herniated disk. As previously explained, when a herniated disc occurs, a small portion of the nucleus pushes out through a tear in the annulus into the spinal canal, allowing the soft, central portion (nucleus) to bulge out. Tears are usually posterior in nature owing to the presence of the posterior longitudinal ligament in the spinal canal. The bulge of a herniated disc causes nerve root compression and spinal stenosis, with resulting pain, and discomfort.

Each vertebra also has two other sets of joints, called facet joints (see FIGS. 2 and 3). The facet joints are located at the back of the spine (posterior). There is one facet joint on each lateral side (right and left). For a given vertebra (e.g., L4), one pair of facet joints faces upward (called the superior articular process) and the other pair of facet joints faces downward (called the inferior articular process). The inferior and superior processes mate, allowing motion (articulation), and link vertebrae together. Facet joints are positioned at each level to provide the needed limits to motion, especially to rotation and to prevent forward slipping (spondylolisthesis) of that vertebra over the one below.

Degenerative changes in the spine can adversely affect the ability of each spinal segment to bear weight, accommodate movement, and provide support. When one segment deteriorates to the point of instability, it can lead to localized pain and difficulties. Segmental instability allows too much movement between two vertebrae. The excess movement of the vertebrae can cause pinching or irritation of nerve roots. It can also cause too much pressure on the facet joints, leading to inflammation.

Facet joint fixation procedures have been used for the treatment of pain and the effects of degenerative changes in the lower back. In one conventional procedure for achieving facet joint fixation, the surgeon works on the spine from the back (posterior). The surgeon passes screws from the spinous process through the lamina and across the mid-point of one or more facet joints.

### II. REPRESENTATIVE SYSTEMS AND METHODS FOR THE BALANCED DISTRACTION AND STABILIZATION OF VERTEBRAE

A. Overview

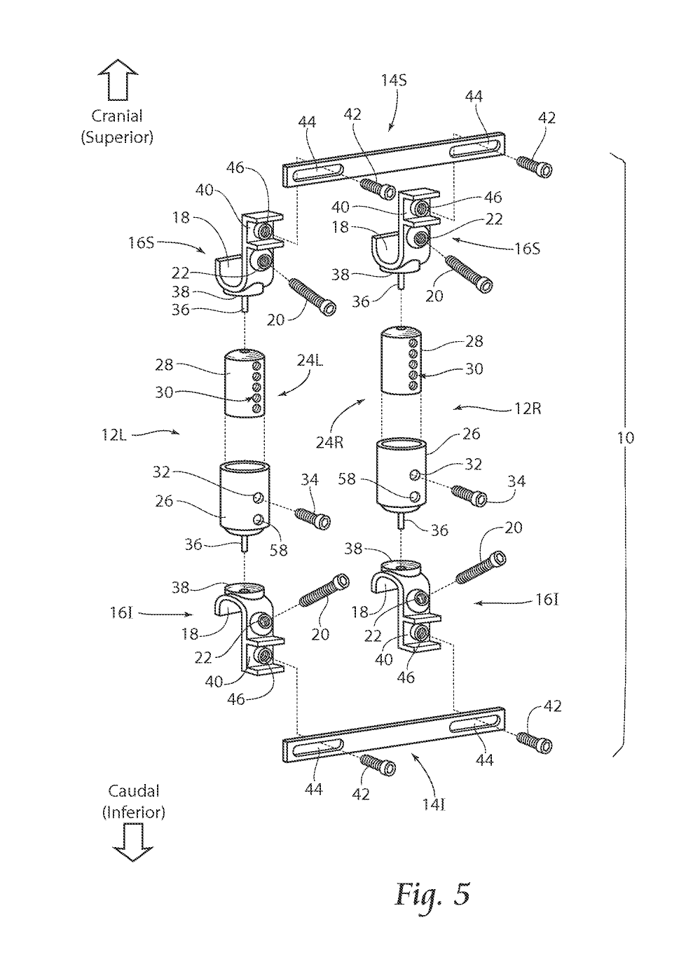

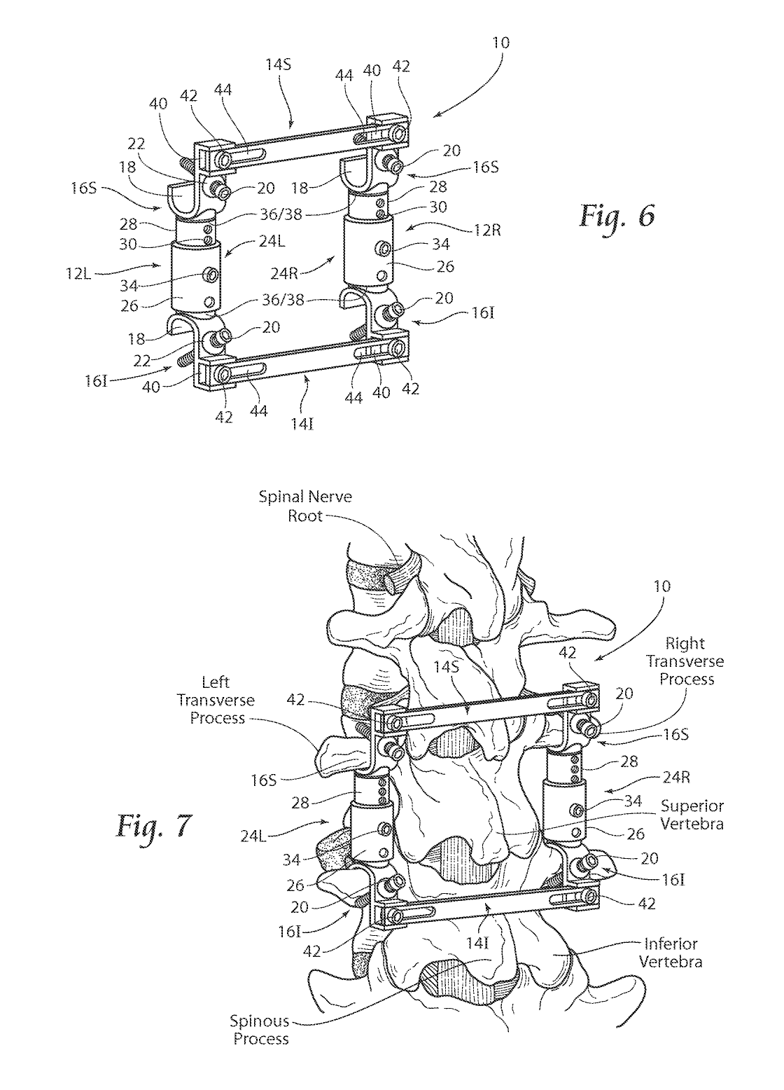

FIG. 5 shows in exploded view a representative system 10 that is sized and configured to be easily assembled (as shown in FIG. 6) and, during assembly, installed (as shown in FIG. 7) between transverse processes of adjacent superior and inferior vertebrae where a herniated disc exists. The superior vertebra is the one closest to the head (or cranial). The inferior vertebra is the one closest to the feet (or caudal).

As shown in FIG. 7, the system 10, when properly assembled and installed, is sized and configured to distract the space between the adjacent vertebrae, providing dynamic stabilization of the vertebrae, which relieves the pressure on the spinal cord and/or nerve roots. When properly assembled and installed, the system 10 indirectly increases the area within the spinal canal, as well as the disc space, thereby, providing an indirect front and back decompression of spinal canal and disc space. As a result, the system 10 can relieve the back and/or leg pain associated with impingement of the nerves due to disc herniation, disc degeneration, scoliosis, post-laminectomy syndrome, and spinal stenosis. The system provides a balanced distraction for both posterior and anterior portions of the spinal column, without causing kyphosis.

FIG. 5 shows the system 10 in an exploded view, prior to assembly and installation, as it exists outside the body. The system 10 comprises a pair of lateral hoist assemblies, respectively 12R and 12L. The lateral hoist assemblies 12R and 12L are sized and configured, when assembled (as FIG. 6 shows), to be mounted, during assembly, on right and left lateral sides of the adjacent vertebrae, between the transverse processes of the adjacent vertebrae, as FIG. 7 shows. In this arrangement, the lateral hoist assemblies 12R and 12L extend generally parallel to the longitudinal axis of the spine.

As FIG. 5 also shows, the system 10 further comprises a pair of transverse superior (or cranial) and inferior (or caudal) brace components, respectively 14S and 14I. The superior and inferior brace components 14S and 14I are sized and configured, when assembled (as FIG. 6 shows), to couple superior and inferior regions of the lateral hoist assemblies 12R and 12L together. When properly assembled and installed (as FIG. 7 shows), the superior and inferior brace components 14S and 14I extending generally transversely across the longitudinal axis of the spine, between the lateral hoist assemblies.

When properly assembled and installed between transverse processes of the adjacent superior and inferior vertebrae, as FIG. 7 shows, the lateral hoist assemblies 12R and 12L exert force upon the transverse processes of the adjacent vertebrae that distracts the space between the adjacent vertebrae. When properly assembled and installed across the lateral hoist assemblies 12R and 12L, as FIG. 7 shows, the superior and inferior brace components 14S and 14I exert a medial force upon the lateral hoist assemblies (i.e., toward the spinous processes of the adjacent vertebrae), to stabilize the lateral hoist assemblies 12R and 12L.

The components 12 and 14 can be made of a durable prosthetic material or composites thereof, such as, e.g., polyethylene, polyether ether ketone (PEEK), rubber, tantalum, titanium, chrome cobalt, surgical steel, ceramic, or an alloy or a combination thereof. PEEK has the advantage of being radiolucent, so it could be x-rayed without any interference from metals, and its material properties are similar to cortical bone, etc.

1. The Hoist Assemblies

Referring to FIGS. 5 and 6, each hoist assembly 12R and 12L, in turn, includes a superior grip element and an inferior grip element, respectively 16S and 16I. The superior grip element 16S is sized and configured, when properly installed (as shown in FIG. 7), to couple to the transverse process of the superior vertebra. Likewise, the inferior grip element 16I is sized and configured, when properly installed (as shown in FIG. 7), to couple to the transverse process of the inferior vertebra.

Each grip element 16S and 16I includes a rest 18 sized and shaped to couple and apply force to a transverse process generally along the longitudinal axis of the spine (i.e., in a superior or inferior direction). As will be exemplified herein, the rest 18 can be variously shaped to achieve this function. As the representative embodiment in FIGS. 5 and 6 shows, the rest 18 is generally j-shaped. The j-shaped rest 18 of the superior grip element 16S is oriented to extend, when properly installed (as FIG. 7 shows), toward the anterior of the superior vertebra, and is further oriented to face in a superior direction. In this way, the j-shaped rest 18 the superior grip element 16S accommodates the application of an upward (cranial) force upon the transverse process of the superior vertebra, as FIG. 7 illustrates.

The j-shaped rest 18 of the inferior grip element 16I is also oriented, when properly installed (as FIG. 7 shows), to extend toward the anterior of the superior vertebra, but is oriented to face in an inferior direction. In this way, the j-shaped rest 18 the inferior grip element 16I accommodates the application of a downward (caudal) force upon the transverse process of the inferior vertebra, as FIG. 7 illustrates, which is opposite to the upward force applied to the transverse process of the superior vertebra. The opposite forces applied by the grip elements 16S and 16I to the transverse processes distract the space between adjacent vertebrae.

Alternative representative embodiments of the rest 18 will be described later.

As FIGS. 5 and 6 show, a gripping screw 20 is threaded through a journal 22 in each j-shaped rest 18. The gripping screw 20 is directed by its respective journal 22 to extend in an oblique path across the j-shaped rest 18, toward and slightly offset beyond the terminus of the j. By loosening the gripping screw 20 (see FIG. 5), the j-shaped rest 18 is opened to accommodate being fitted to its respective transverse process during installation, or (if desired) released from the transverse process. By tightening the gripping screw 20 (see FIGS. 6 and 7), the gripping screw 20 closes the j-shaped rest 18, to confine the respective transverse process within the interior of the j-shaped rest 18 (as FIG. 7 shows). The gripping screw 20 and j-shaped rest 18 together serve to releasably couple the j-shaped rest 18 (and therefore the hoist assemblies 12R and 12L themselves) to the transverse process without the need to pass the screw 20 into cortical bone itself.

As FIGS. 5 and 6 further show, each hoist assembly 12R and 12L further includes an adjustable support column, respectively 24R and 24L. The support columns members 24R and 24L are coupled to the superior and inferior grip elements 16S and 16I, and extend, respectively, between the transverse processes on the right and left sides of the adjacent vertebrae. The adjustable support columns 24R and 24L are sized and configured to be adjusted to generate and maintain the upward and downward forces applied by the j-shaped rests 18 to the transverse processes, as just described and as shown in FIG. 7. It is these upward and downward forces, simultaneously applied by the support columns 24R and 24L to the j-shaped rests 18, that distract the space between the adjacent vertebrae.

The support columns 24R and 24L can be variously shaped and configured. The support columns 24R and 24L in cross section can, e.g., be generally curvilinear (i.e., round or oval) or be generally rectilinear (i.e., square or rectangular or hexagon or H-shaped or triangular), or combinations thereof. The cross section of a given support column 24R and 24L can be generally uniform, or it can vary along its length (e.g., taper).

In a representative embodiment (see in particular FIG. 5), each adjustable support column 24R and 24L comprises a curvilinear cylinder component 26 and a rod component 28. The rod component 28 is concentrically carried within the cylinder component 26 for advancement axially into and out of the cylinder component 26, thereby axially lengthening or shortening the overall length of the respective support column 24R or 24L. The longer the overall length of the support column 24R or 24L, the greater the magnitude of the upward and downward forces applied to the j-shaped rests 18 to distract the space between the adjacent vertebrae.

In the illustrated embodiment, the cylinder component 26 is inferior to the rod component 28. In this arrangement, the grip element 16S is coupled to the rod component 28, and the grip element 16I is coupled to the cylinder component 26. Preferable, as FIG. 5 shows, the coupling is not rigid, but provides a compliant junction between the support column and the grip elements that accommodates native movement between the adjacent vertebrae. The coupling can include, e.g., a pivot pin 36 that accommodates relative rotational movement between the grip element 16S and 16I and its respective support column component 26 and 28. Alternatively, or in combination, the coupling can comprise an elastomeric material 38 that provides compliance at the junction between the support column and the grip elements.

It should be appreciated that the relative orientation of the cylinder and rod components 26 and 28 (superior vs. inferior) is not believed to be critical and can accordingly be reversed.

In the illustrated embodiment (see FIG. 5), the rod component 28 includes a linear array of threaded journals 30, axially spaced apart along one side of the component 28. In this arrangement, the cylinder component 26 includes an aperture 32 that will come into successive registration with one of the threaded journals 30 as the rod component 28 is successively moved within the cylinder component 26 to adjust the length of the respective support column 24R and 24L.

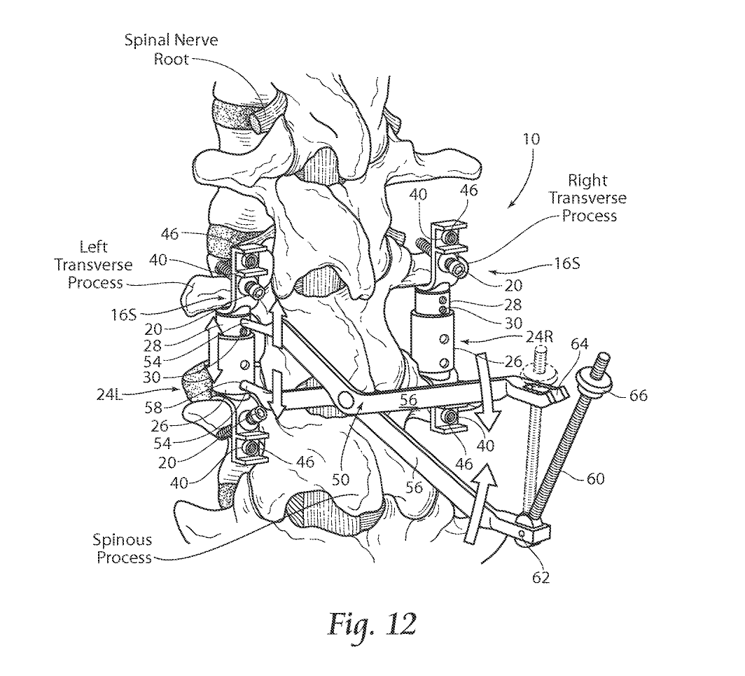

As will be described in greater detail later, the system 10 can include a column adjustment tool 50 (see FIG. 11) to apply a mechanical advantage when adjusting the length of the respective support column 24R and 24L (shown in FIG. 12).

As FIGS. 5 and 6 show, the each hoist assembly 12R and 12L includes a locking screw 34. The locking screw 34 is sized and configured to be passed through the aperture 32 and threaded into the journal 30 that is in then-current registry with the aperture 32. The locking screw 34 secures the then-current position of the rod component 28 and thereby fixes the then-current length of the respective support column 24R and 24L.

The grip elements 16S and 16I (see FIGS. 5 and 6) include brace attachment sites 40 sized and configured to receive and secure the respective superior and inferior brace components 14S and 14I. In the illustrated embodiment, the attachment sites 40 take the form of brackets formed above the leg j-shaped rest 18.

2. The Brace Components

As FIG. 5 further shows, opposite left and right ends of the superior brace component 14S are sized and configured to nest within the attachment sites 40, left and right, on the superior j-shaped rests 18 (on the superior vertebra). When properly assembled and installed (see FIGS. 6 and 7), the superior brace component 14S is oriented to laterally span between the left and right transverse processes of the superior vertebra above (cranial to) the intermediate spinous process. In the illustrated embodiment, locking screws 42 pass through slotted openings 44 in the left and right ends of the superior brace component 14S and into a raised, threaded journal 46 that projects in a posterior direction within the attachment sites 40. The raised journal 46 offsets the superior brace component 14S in a posterior direction to accommodate the native curve of the vertebral body between the transverse processes above (cranial to) the intermediate spinous process spanned by the superior brace component 14S. The locking screw 42 secures the superior or cranial brace component 14S to the superior grip elements 16S engaging the transverse processes of the superior (or cranial) vertebra.

Likewise, as FIG. 5 also shows, opposite left and right ends of the inferior brace component 14I are sized and configured to nest within the attachment sites 40, left and right, on the inferior j-shaped rests 18 (on the inferior vertebra). When properly assembled and installed (see FIGS. 6 and 7), the inferior brace component 14I is oriented to laterally span between the left and right transverse processes of the inferior vertebra above (cranial to) the intermediate spinous process. In the illustrated embodiment, locking screws 42 pass through slotted openings 44 in the left and right ends of the inferior brace component 14I and into a raised, threaded journal 46 that projects in a posterior direction within the attachment sites 40. The raised journal 46 offsets the inferior brace component 14I in a posterior direction to accommodate the native curve of the vertebral body between the transverse processes above (cranial to) the intermediate spinous process spanned by the inferior brace component 14I. The locking screw 42 secures the inferior or caudal brace component 14I to the inferior grip elements 16I engaging the transverse processes of the inferior (or caudal) vertebra.

The superior and inferior brace components 14S and 14I are preferably assembled to the grip elements 16S and 16I to exert a medial force upon the lateral hoist assemblies 12R and 12L (i.e., toward the spinous processes of the adjacent vertebrae), to stabilize the lateral hoist assemblies 12R and 12L. As will be described in greater detail later, the system 10 can include a brace adjustment tool 52 (see FIG. 15) to provide a mechanical advantage when creating the medial force during the assembly of the brace components 14S and 14I to the grip elements 16S and 16I.

B. Assembly and Installation

Prior to assembly and installation of the representative system 10, the location of a herniated vertebral disc between adjacent vertebrae is identified. The system 10 can be assembled and installed in situ using, e.g., a conventional open posterior—from the back—surgical approach to the adjacent vertebrae that are affected.

1. Installation of the Hoist Assemblies

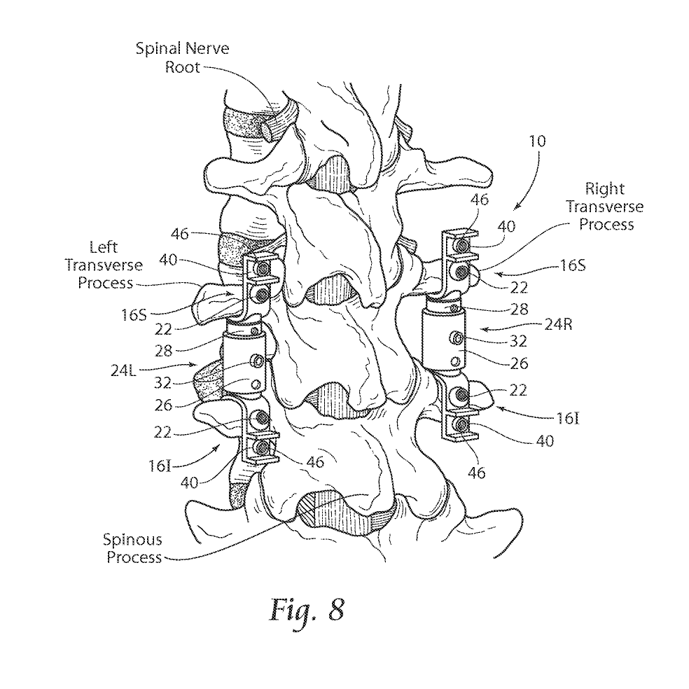

During initial installation of the system 10, see FIG. 8, the length of the support columns 24R and 24L is originally reduced and locked by the locking screw 34 at a length that is less than the existing distance between the transverse processes of the adjacent vertebrae. This allows initial fitment of the j-shaped rests 18 to their respective transverse processes, as FIG. 8 shows.

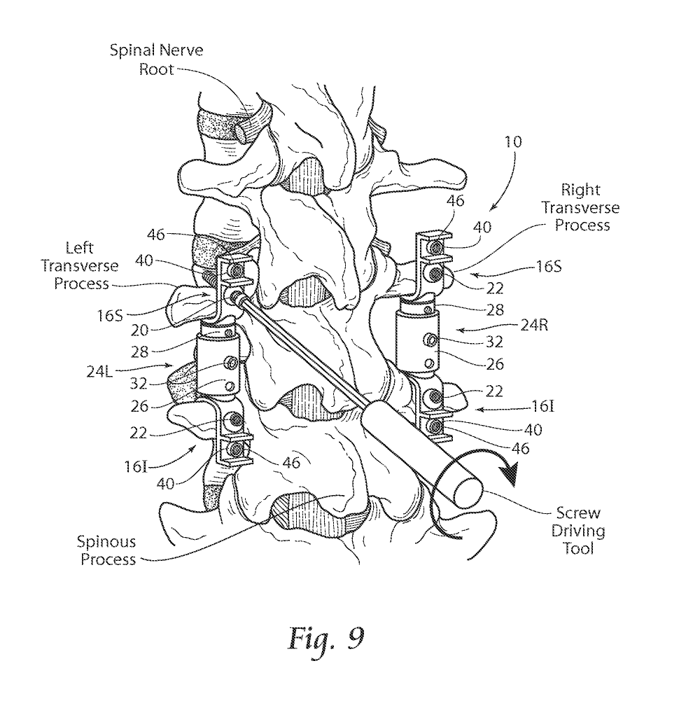

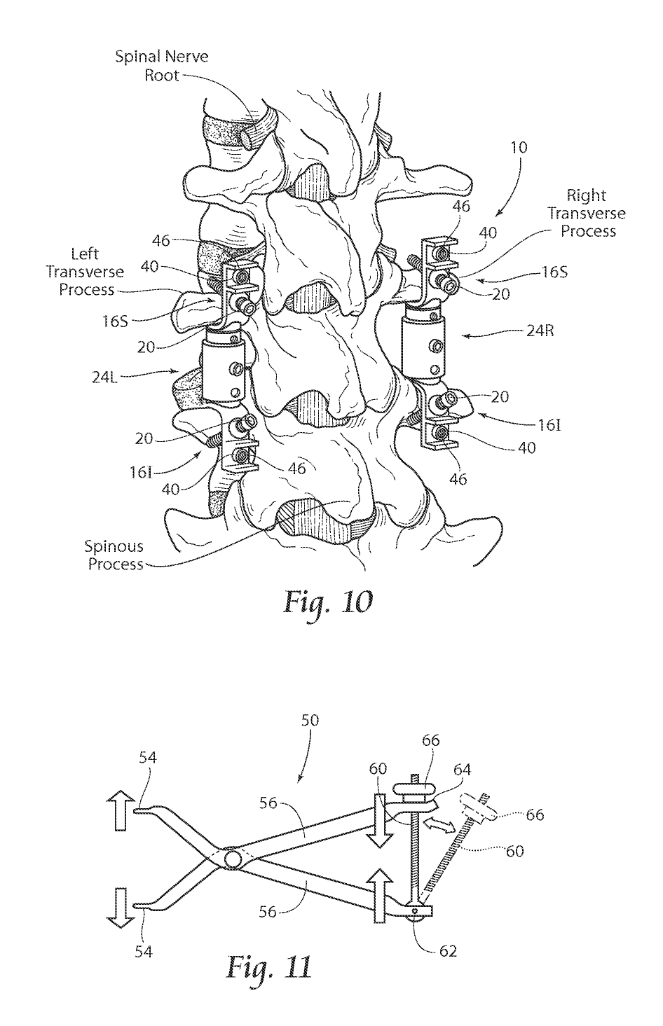

The gripping screws 20 are inserted by a suitable screw-driving tool (see FIG. 9) into the j-shaped rests 18 to close the j-shaped rests 18 and install the hoist assemblies 12R and 12L in the existing space between the transverse processes of the adjacent vertebrae (see FIG. 10).

As FIG. 12 shows, the locking screws 34 are loosened (or removed), and the rod component 28 is moved outward of the cylinder component 26 of the respective support column 24R and 24L, to increase the length of the respective support column 24R and 24L, in succession right and left, between the transverse processes. Lengthening the support columns 24R and 24L exerts an inferior-superior separating force to the transverse processes. The inferior-superior separating force on the transverse processes in turn increases the distance between the adjacent vertebrae. The separating force is applied until a desired distance between the adjacent vertebrae is achieved, i.e., when the desired therapeutic benefit is achieved, e.g., to relieve pressure on the spinal cord and/or nerve roots occasioned by herniated condition of the disc.

As FIG. 12 shows, a column adjustment tool 50 (shown in FIG. 11) is desirably used to provide a mechanical advantage while adjusting the length of the respective support column 24R and 24L, until a desired distance between the adjacent vertebrae is achieved. In the illustrated embodiment (see FIG. 11), the column adjustment tool 50 comprises a pair of grippers 54 at the distal ends of lever arms 56 coupled at a pivot 56. As shown in FIG. 12, the inferior gripper 54 engages a gripper aperture 58 provided on the inferior region of the cylinder component 26, and the superior gripper 54 engages the most superior journal 30 on the rod component 28. By applying hand pressure to squeeze the proximal ends of the lever arms together, the grippers 54 at the distal end separate, to increase the length of the respective support column 24R and 24L and apply the separating force.

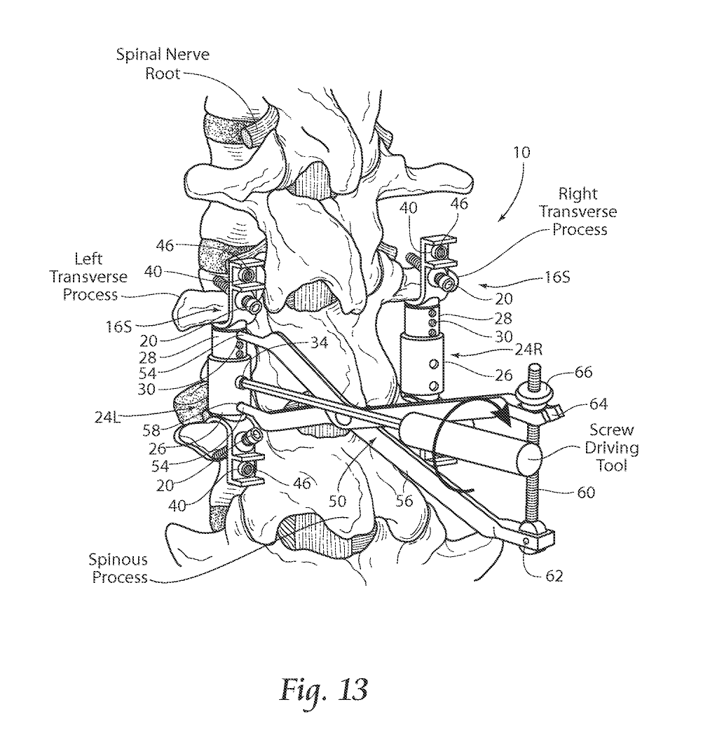

A threaded bar 60 attached by a pivot 62 on the proximal end of one of the lever arms 56 swings into and out of a U-shaped slot 64 formed on the proximal end of the proximal end of the other lever arm 56. An enlarged stop nut 66 is threaded on the free end of the bar 58. The bar 58 is swung free of the slot 64 while the proximal ends of the lever arms are squeezed together (as shown in solid lines in FIG. 12), to apply the separating force. When the desired distance between the adjacent vertebrae is achieved, the bar 60 is swung into the slot 64 and the stop nut 66 tightened (as shown in phantom lines in FIG. 12 and in solid lines in FIG. 13) to maintain the separating force while the locking screw 34 is tightened (by a suitable screw-driving instrument) to secure the then-current position of the rod component 28 and thereby fix the then-current length of the respective support column 24R and 24L, as is shown in FIG. 13. The lengths of both support columns 24R and 24L are adjusted in this manner to achieve the desired distance, and the locking screws 34 are tightened to lock the support columns 24R and 24L at the distance that maintains the desired distance (as FIG. 14 shows). Balanced, left and right lateral side adjustment of the support columns 24R and 24L is thereby accomplished. The stop nut 66 can then be loosened and the column adjustment tool 50 removed.

2. Installation of the Brace Components

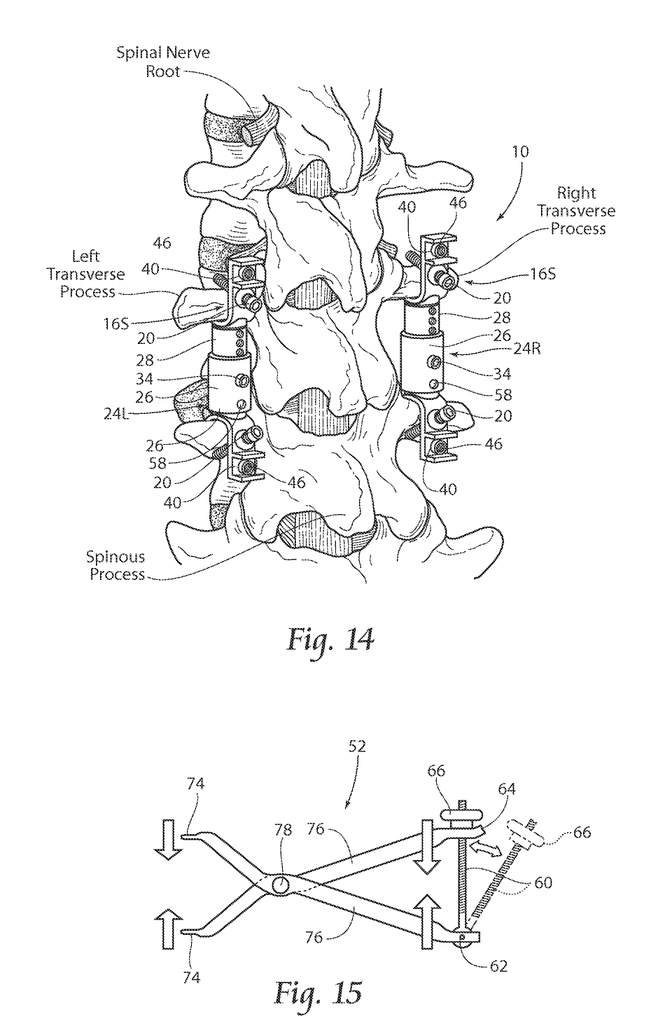

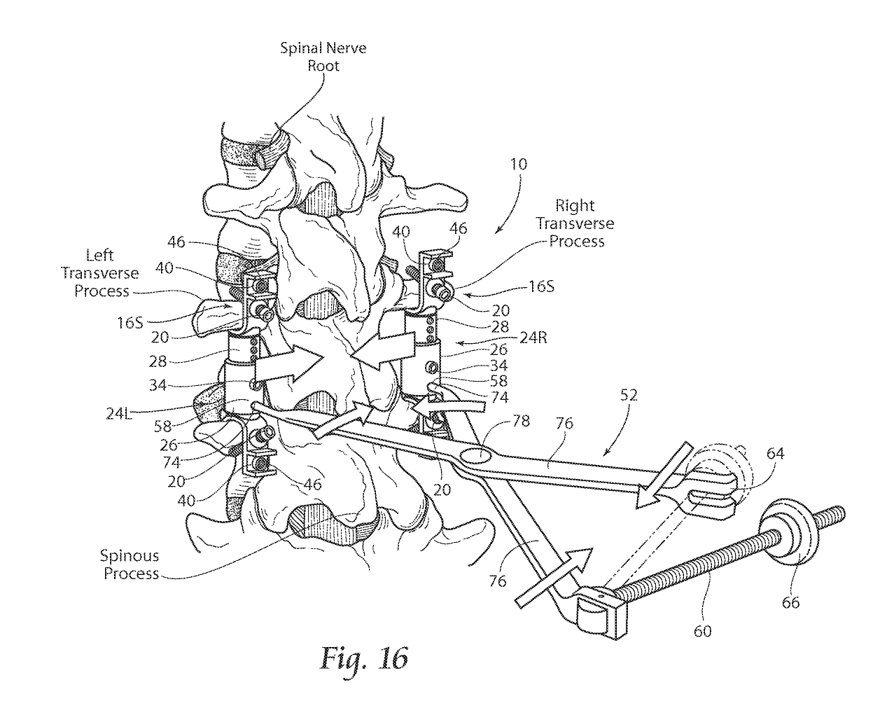

The superior and inferior brace components 14S and 14I are preferably then assembled to the grip elements 16S and 16I. As previously stated, a brace adjustment tool 52 (see FIG. 15) can be used to provide a mechanical advantage to create a medial force during the assembly of the brace components 14S and 14I to the grip elements 16S and 16I. In the illustrated embodiment, the brace adjustment tool 52 (see FIG. 15) comprises a pair of grippers 74 at the distal ends of lever arms 76 coupled at a lazy-tongs linkage 78, like a scissor. As shown in FIG. 16, the grippers 74 mutually engage the gripper apertures 58 provided on the inferior regions of the cylinder components 26, left and right. By applying hand pressure to squeeze the proximal ends of the lever arms 76 together, the grippers 74 move toward each other, to apply a medial force toward the spinous process between the respective support columns 24R and 24L.

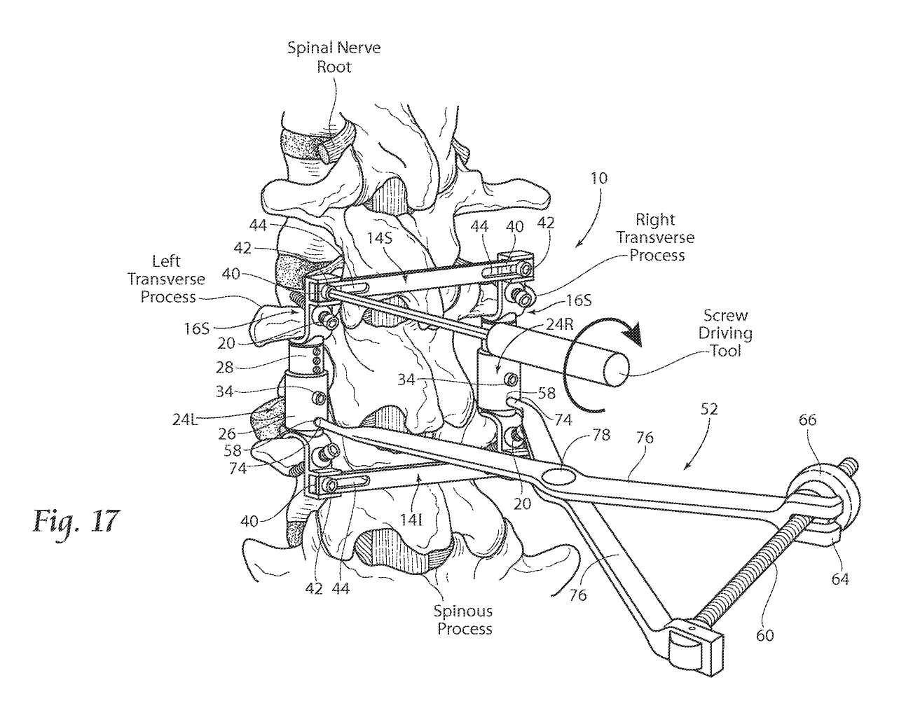

As on the column adjustment tool 50, the bar adjustment tool 52 includes a threaded bar 60 attached by a pivot 62 on the proximal end of one of the lever arms 76 of the brace adjustment tool 52. The threaded bar 60 swings into and out of a U-shaped slot 64 formed on the proximal end of the proximal end of the other lever arm 76. An enlarged stop nut 66 is threaded on the free end of the bar 60. The bar 60 is swung free of the slot 64 while the proximal ends of the lever arms 76 are squeezed together (as shown in solid lines in FIG. 16), to apply the medial force. When the desired transverse distance between the support columns 24R and 24L is achieved, the bar 60 is swung into the slot 64 and the stop nut 66 tightened (as shown in phantom lines in FIG. 16 and in solid lines in FIG. 17) to maintain the medial force while the opposite left and right ends of the superior brace component 14S and inferior brace components 14I are secured by the locking screws 42 to the superior and inferior grip elements 16S and 16I. The stop nut 66 can then be loosened and the column adjustment tool 52 removed.

3. Benefits of the System

The system 10 serves to distract the space between the adjacent vertebrae, providing dynamic stabilization of the vertebrae to relieve the pressure on the spinal cord and/or nerve roots. The distraction of the transverse processes enlarges the volume of the spinal canal to alleviate pressure on blood vessels and/or nerves, thereby treating the pain and other symptoms that can accompany disc herniation and/or spinal stenosis. The system 10 provides an indirect front and back decompression of spinal canal and disc space. As a result, the system 10 can serve to relieve the back and/or leg pain associated with impingement of the nerves due to disc herniation, disc degeneration, scoliosis, post-laminectomy syndrome, and spinal stenosis. The system 10 provides a balanced distraction for both posterior and anterior portions of the spinal column, without causing kyphosis.

After installation, the system 10 also serves as a dynamic stabilizer for the back. As the back is bent backwardly and placed in extension, or forwardly and placed in flexion, the presence of the system 10 resists extension and flexion beyond a given point. Due to the presence of the system 10, the spacing between adjacent transverse processes cannot be reduced to less than the desired spacing established by the support columns. Pressure on nerves and the resulting pain are therefore alleviated or reduced.

C. Posterolateral Fusion

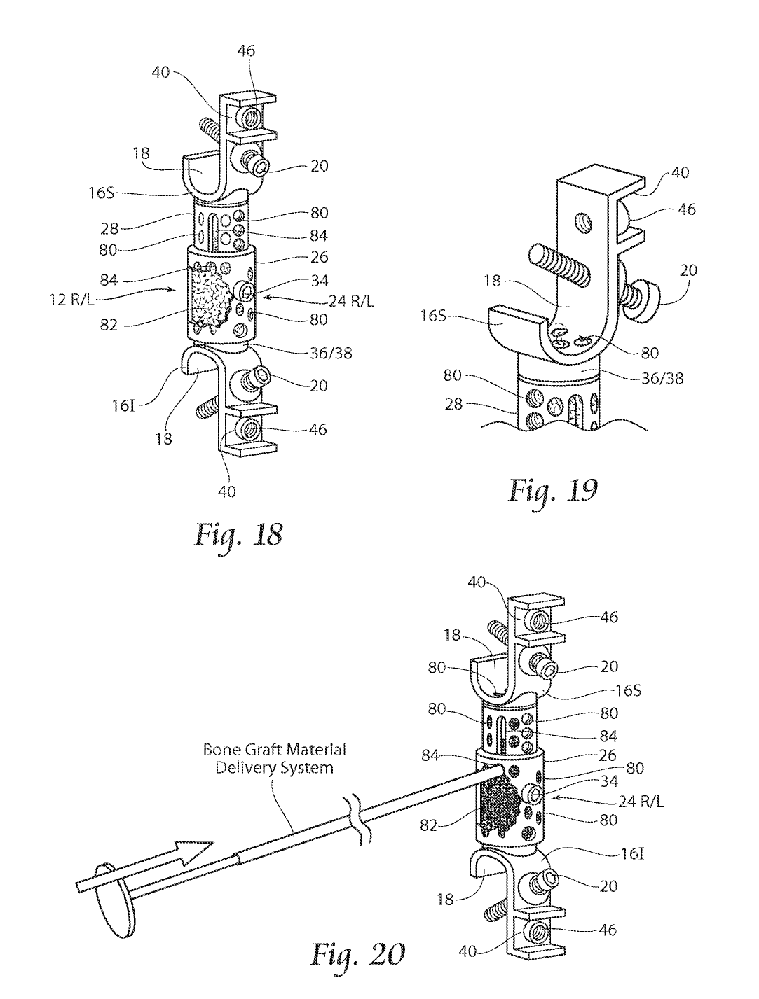

The system 10 can also be adapted to achieve posterolateral spinal fusion between adjacent vertebrae, without fusion within the disc space itself. As shown in FIG. 18, the cylinder component 26 and the rod component 28 of the support columns 24R and 24L can be modified to include an array of holes or fenestrations 80 and to accommodate inside the cylinder component 26 and rod component 28 the placement of bone graft material 82. Desirably (see FIG. 19), the inner curve of the U-shaped rests 18 also includes holes or fenestrations 80 that communicate with the interior of the adjoining cylinder component 26 or rod component 28, to allow the bone graft material 82 to extend to the transverse process.

The system 10 including the fenestrated support columns 24R and 24L, desirably containing an initial volume of bone graft material 82, is installed and assembled in the manner previously described. Once the system 10 is assembled and installed, and the desired distance between the adjacent vertebrae is achieved and locked, followed by the achieving and locking the desired medial stabilization force, additional bone graft material can be packed into the cylinder component 26 and/or the rod component 28 to complete the installation, as shown in FIG. 20. For this purpose, the cylinder component 26 and/or the rod component 28 can include elongated bone graft introduction sites 84, through which bone graft material 82 can be introduced by a suitable bone graft delivery system, e.g., by manual tamping through a delivery cannula, or by low-pressure injection by syringe or the like.

The bone graft material sets to a hardened condition within the cylinder component 26 and rod component 28, as well as within the curve of the j-shaped rests 18. A “posterolateral fusion” is achieved by a fused distraction between transverse processes, without contiguous fusion of the disc between the adjacent vertebrae. The posterolateral fusion further separates and holds two vertebrae apart, to make the opening around the nerve roots bigger and relieving pressure on the nerves. As the vertebrae separate, the ligaments tighten up, reducing instability and mechanical pain.

### III. OTHER REPRESENTATIVE SYSTEMS AND METHODS FOR THE BALANCED DISTRACTION AND STABILIZATION OF VERTEBRAE

A. Balanced Distraction

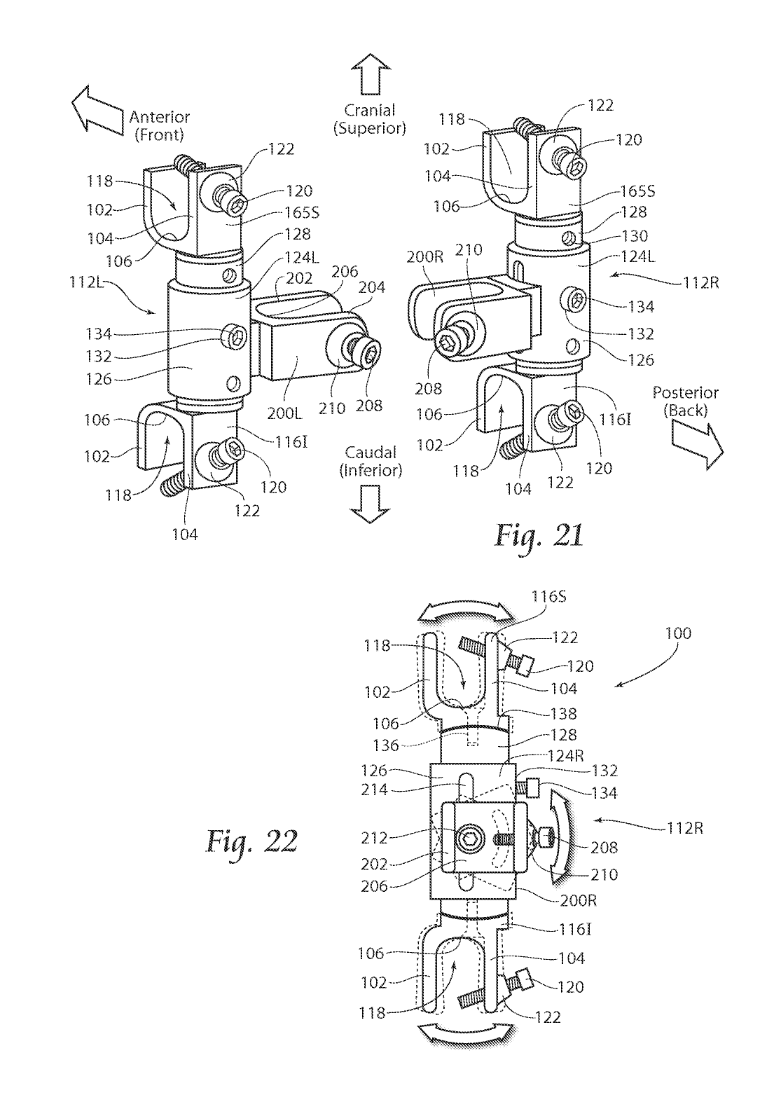

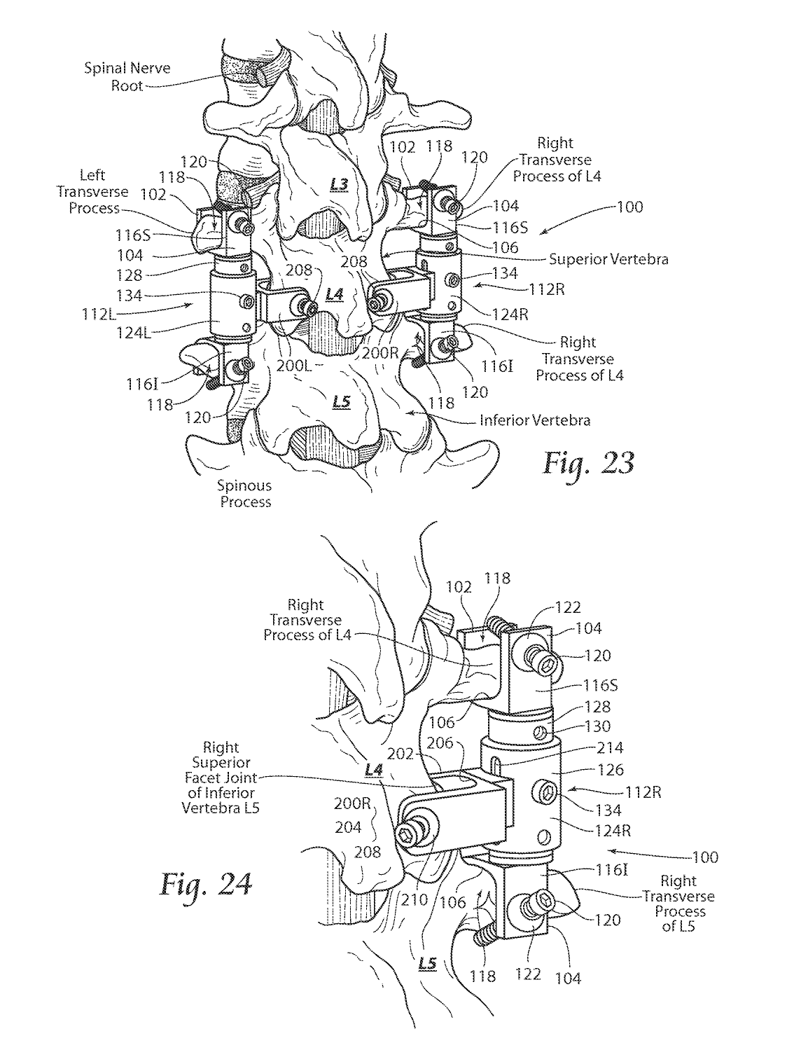

FIGS. 21 and 22 show another representative system 100 that is sized and configured to be easily assembled and installed (as shown in FIG. 23) between transverse processes of adjacent superior and inferior vertebrae where a herniated disc exists. The representative system 100 includes many of the basic components of the representative system 10, previously described and shown in FIG. 6. However, the system 100 includes additional technical features, as will now be described.

Like the representative system 10, the representative system 100 includes a pair of lateral hoist assemblies 112R and 112L. As previously described, the lateral hoist assemblies 112R and 112L are sized and configured to be mounted on right and left lateral sides of the adjacent vertebrae, between the transverse processes of the adjacent vertebrae, as FIGS. 23 and 24 further show. In this arrangement, the lateral hoist assemblies 112R and 112L extend generally parallel to the longitudinal axis of the spine (see FIG. 23), in the same manner as the hoist assemblies 12L and 12R do in the representative system 10.

Like the representative system 10, each hoist assembly 112R and 112L, in turn, includes a superior grip element and an inferior grip element, respectively 116S and 116I. The superior grip element 116S is sized and configured, when properly installed (as shown in FIGS. 23 and 24), to couple to the transverse process of the superior vertebra. Likewise, the inferior grip element 116I is sized and configured, when properly installed (as also shown in FIGS. 23 and 24), to couple to the transverse process of the inferior vertebra.

As in the representative system 10, each grip element 116S and 116I of the representative system 110 further includes a rest 118 shaped to couple and apply force to a transverse process generally along the longitudinal axis of the spine. In the system 100, the rests 118 are sized and configured differently than the rests 18 in the system 10.

More particularly, each rest 118 in the system 110 comprises a more symmetric “u-shape,” compared to the less symmetric “j-shaped” rests 18 of the system 10. The symmetric u-shape of each rest 118 in the system 110 comprises anterior-facing sidewall 102 and a posterior-facing sidewall 104 separated by a base wall 106, which in the illustrated embodiment is chamfered or curved. In this more symmetric arrangement, the anterior-facing and posterior-facing sidewalls 102 and 104 possess generally equal heights, as measured along the longitudinal axis of the companion hoist assembly 112R and 112L. The heights are selected to at least correspond to the inferior-to-superior dimensions of a typical transverse process. The separation afforded by the curved base wall 106 is also selected to corresponding to the anterior-to-posterior dimensions of a typical transverse process.

The morphology of the local structures can be generally understood by medical professionals using textbooks of human skeletal anatomy along with their knowledge of the site and its disease or injury. The physician is also able to ascertain the dimensions of the rests 118 based upon prior analysis of the morphology of the targeted bone region using, for example, plain film x-ray, fluoroscopic x-ray, or MRI or CT scanning.

As FIG. 24 illustrates, the anterior-facing and posterior-facing sidewalls 102 and 104 and the curved base wall 106 together form a symmetric rest 118, which wholly captures, in a non-traumatic manner, a given transverse process on anterior, posterior, and inferior/superior anatomic sides. The symmetry of the rests 118 resists both anterior and posterior translation of the transverse process within the pocket region, lending stability to the overall system 100.

Within this construct, the u-shaped rests 118 of the superior grip element 116S are oriented to face in a superior direction, with the longitudinal axis of the rest 118 in alignment with the lateral axis of the transverse process of the superior vertebra (see FIGS. 23 and 24). Conversely, the u-shaped rests 118 of the inferior grip element 116I are oriented to face in an inferior direction, with the longitudinal axis of the rest 118 in alignment with the lateral axis of the transverse process of the inferior vertebra (see FIGS. 23 and 24).

As FIGS. 21 and 22 show, as in the system 10, the system 110 includes a gripping screw 120 threaded through a journal 122 in each u-shaped rest 118. The gripping screw 120 is directed by its respective journal 122 (see FIG. 22) to extend in an oblique path from the posterior facing sidewall 104 across the u-shaped rest 118 toward and terminating in a slightly offset relationship with the anterior-facing sidewall 102. The gripping screw 120 captures the respective transverse process within the rest 118 (see FIGS. 23 and 24).

As previously described with respect to the system 10, the gripping screw 120 of the system 110 can be unscrewed to open the u-shaped rest 118 and accommodate being fitted to its respective transverse process during installation, or (if desired) released from the transverse process. The gripping screw 120 of the system 110 can be tightened to close the u-shaped rest 118, to capture the respective transverse process within the interior of the u-shaped rest 118.

The gripping screw 120 and u-shaped rest 118 together serve to releasably couple the u-shaped rest 118 (and therefore the hoist assemblies 112R and 112L themselves) to the transverse process without the need to pass the screw 120 into cortical bone itself.

As FIGS. 21 and 22 show, each hoist assembly 112R and 112L of the system 110 further includes an adjustable support column, respectively 124R and 124L, coupled to the superior and inferior grip elements 116S and 116I. As in the system 10, the support columns 124R and 124L comprise curvilinear cylinder components 126 and mating rod components 128. The rod components 128 are concentrically carried within the cylinder components 126 for advancement axially into and out of the cylinder component 126, thereby axially lengthening or shortening the overall length of the respective support column 124R or 124L.

As previously described with respect to the system 10, the rod components 128 of the system 110 include a linear array of threaded journals 130 (also shown aa journals 30 in FIG. 5), axially spaced apart along one side of the component 128. In this arrangement, the cylinder components 126 include an aperture 132 that will come into successive registration with one of the threaded journals 130 as the rod component 128 is successively moved within the cylinder component 126 to adjust the length of the respective support column 124R and 124L.

Each hoist assembly 112R and 112L of the system 110 includes a locking screw 134. The locking screw 134 is sized and configured to be passed through the aperture 132 and threaded into the journal 130 that is in then-current registry with the aperture 132. The locking screw 134 secures the then-current position of the rod component 128 and thereby fixes the then-current length of the respective support column 124R and 124L.

In the system 110 (see FIGS. 23 and 24), the u-shaped rest 118 the superior grip element 116S applies an upward (cranial) force upon the transverse process of the superior vertebra, its symmetric shape resisting either anterior or posterior slippage. As FIGS. 23 and 24 also show, the u-shaped rest 118 the inferior grip element 116I applies a downward (caudal) force upon the transverse process of the inferior vertebra, its symmetric shape also resisting either anterior or posterior slippage. The opposite forces applied by the grip elements 116S and 116I to the transverse processes distract the space between adjacent vertebrae. The longer the established overall length of the support column 124R or 124L, the greater the magnitude of the forces applied to the u-shaped rests 118.

To complement an anatomical alignment between the rests 118 and the respective transverse process, and thereby provide greater stability (as is best shown in FIG. 22), the inferior-superior axis of each u-shaped rest 118 is desirably offset in an anterior direction from the longitudinal axis of the respective hoist assembly 112R and 112L, In this arrangement, the most anterior-facing sidewall of each rest 118 projects beyond or overhangs the anterior side of the respective hoist assembly 112R and 112L.

To further enhance the form and fit of the anatomical alignment between the u-shaped rests 118 and the respective transverse process (see FIG. 22), each rest 118 can be mounted via a pivot pin 136 on a convex bearing surface 138 on the inferior and superior end of the adjustable support columns 1124R and 1124L. This pivoting linkage is sized and configured to accommodate relative movement between the rest 118 and its respective hoist assembly (as shown by phantom lines and arrows in FIG. 22), to conform to the particular anatomy and to the dynamics of the in situ forces encountered. In the illustrated embodiment, at least three degrees of movement are accommodated; namely, rotation of the u-shaped rest 118 about the longitudinal axis of the respective support column 112R and 112L; anterior-to-posterior rocking motion of the u-shaped rest 118 relative to the respective support column 112R and 112L; and medial-to-lateral rocking motion of the u-shaped rest 118 relative to its respective support column 112R and 112L. The degree of rotational, anterior-to-posterior, and medial-to-lateral movement between the rest 118 and its respective support column 112R and 112L is desirably mechanically limited (e.g., by plus or minus 5° from a neutral central position). However, within the mechanical limits imposed, an infinite range of movement 360° about neutral central position is desirably accommodated.

B. Balanced Distraction with Facet Joint Fixation

The system 110 shown in FIGS. 21 and 22 provides additional stabilization between adjacent targeted vertebrae, by making possible the fixation of the facet joints between the adjacent vertebrae. In the representative embodiment shown in FIGS. 21 and 22, each pair of lateral hoist assemblies 112R and 112L includes a facet joint fixation bracket 200R and 200L. As shown in FIGS. 23 and 24, in use at a given vertebral level (which for the purpose of illustration in FIGS. 23 and 24 comprises a superior vertebra (L4) and an inferior vertebra (L5) that have been distracted by a hoist assembly 112R/112L), the brackets 200R and 200L are sized and configured to mutually capture, on right and left lateral sides, the superior articular facet joints of the inferior vertebra (L5), to thereby fixate right and left superior facet joints of L5 at that level (stated differently, from a different perspective, the brackets 200R and 200L fixate the right and left lateral inferior articular facet joints of the superior vertebra (L4)).

For this purpose (as FIGS. 23 and 24 show), a given facet joint fixation bracket 200R and 200L is cantilevered in a medial direction from its respective hoist assembly 112R and 112L toward the facet joint most adjacent the inferior grip element 116R/L. That is, for a right hoist assembly 112R (see FIG. 24), the facet joint fixation bracket 200R is cantilevered medially in a left direction toward the right superior facet joint of the inferior vertebra (which in FIG. 24 is L5). Conversely, for a left hoist assembly 112L (see FIG. 23), the facet joint fixation bracket 200L is cantilevered medially in a right direction toward the right superior facet joint of the inferior vertebra (L5).

The right and left facet joint brackets 200R and 200L can be variously constructed. In the representative embodiment shown in FIGS. 21, 22, and 24, each bracket 200R and 200L includes medially extending, symmetric walls comprising am anterior-facing wall 202 and posterior-facing wall 204. The anterior-facing and posterior-facing walls 202 and 204 are joined by an intermediate base wall 206, which in the illustrated embodiment is chamfered or curved. The longitudinal axis of each bracket 200R and 200L is generally normally aligned with the longitudinal axis of the companion hoist assembly 112R and 112L. This orientation allows (with reference to FIG. 24), using a posterior approach, the brackets 200R and 200L to be slid in a medial direction over the intended facet joint, as the rests 118 of the companion hoist assemblies 112R and 112L are slid in superior and inferior directions onto the transverse processes (the installation of the hoist assemblies 12R and 12L of the system 10 has been previously described, and the same installation techniques can be used to install the system 110, as well).

In the representative embodiment (see FIGS. 21 and 22), the anterior-facing and posterior-facing walls 202 and 204 possess generally equal superior-inferior heights and medial lengths, measured, respectively, along and from the longitudinal axis of the companion hoist assembly 112R and 112L. The heights are selected to at least correspond to the inferior-to-superior dimensions of a typical facet joint. The medial length is selected to at least correspond to the medial distance between a typical transverse process and most adjacent facet joint. The separation afforded by the curved base wall 206 is also selected to corresponding to the anterior-to-posterior dimensions of the facet joint.

The morphology of the local structures can be generally understood by medical professionals using textbooks of human skeletal anatomy along with their knowledge of the site and its disease or injury. The physician is also able to ascertain the dimensions of the brackets 200R and 200L based upon prior analysis of the morphology of the targeted bone region using, for example, plain film x-ray, fluoroscopic x-ray, or MRI or CT scanning.

The anterior-facing and posterior-facing walls 202 and 204 and the curved base wall 206 together form a symmetric bracket 200R or 200L that wholly captures, in a non-traumatic manner, a facet joint between given superior and inferior vertebra.

As FIGS. 21 and 22 show, the system 110 includes a gripping screw 208 threaded through a journal 210 in each bracket 200R and 200L. The gripping screw 208 is directed by its respective journal 204 (see FIG. 22) to extend in an oblique path, medial to lateral, from the posterior facing wall 204 across the bracket 200R and 200L toward and terminating in a slightly offset relationship with the anterior-facing wall 202.

The gripping screw 208 can be unscrewed to open the bracket 200R and 200L to accommodate fitment to its respective facet joint during installation, or (if desired) released from the facet joint. The gripping screw 208 can be tightened to close the bracket 200R and 200L, to capture the respective facet joint within the interior of the bracket 200R and 200L. The gripping screw 208 captures the facet joint within the bracket 200R and 200L and thereby fixates the facet joint.

The gripping screw 208 and the bracket 200R and 200L together serve to releasably couple the bracket 200R and 200L (and therefore the companion hoist assemblies 112R and 112L themselves) to the facet joint without the need to pass the screw 208 into cortical bone itself.

To complement anatomical alignment between the brackets 200R and 200L and the respective facet joint, and thereby provide greater stability (as is best shown in FIG. 22), the inferior-superior axis of each bracket 200R and 200L is desirably offset in an posterior direction from the longitudinal axis of the respective hoist assembly 112R and 112L, In this arrangement, the most posterior-facing wall 204 of each bracket 200R and 200L projects beyond or overhangs the posterior side of the respective hoist assembly 112R and 112L.

To further enhance the form and fit of the anatomical alignment between the brackets 200R and 200L and the respective facet joint, each bracket 200R and 200L can be mounted via a pivot pin 212 within a track 214 formed along its companion cylinder component 126. This provides a linkage that permits pivoting as well as sliding the bracket 200R and 200L along the longitudinal axis to accommodate relative movement between the bracket 200R and 200L and its respective hoist assembly 112R and 112L, to conform to the particular anatomy and to the dynamics of the in situ forces encountered.

As shown in FIGS. 23 and 24, for a given lateral hoist assembly 112R and 112L, the respective facet joint fixation bracket 200R and 200L is sized and configured to receive and fuse the respective right or left superior facet joint of the inferior vertebra in tandem with the distraction provided by the lateral hoist assemblies 112R and 112L. In this way, the two functional components of the system 100 provide dynamic stabilization of the vertebrae and facet joints to relieve the pressure on the spinal cord and/or nerve roots and attendant pain.

C. Balanced Distraction with Facet Joint Fixation and Medial Bracing

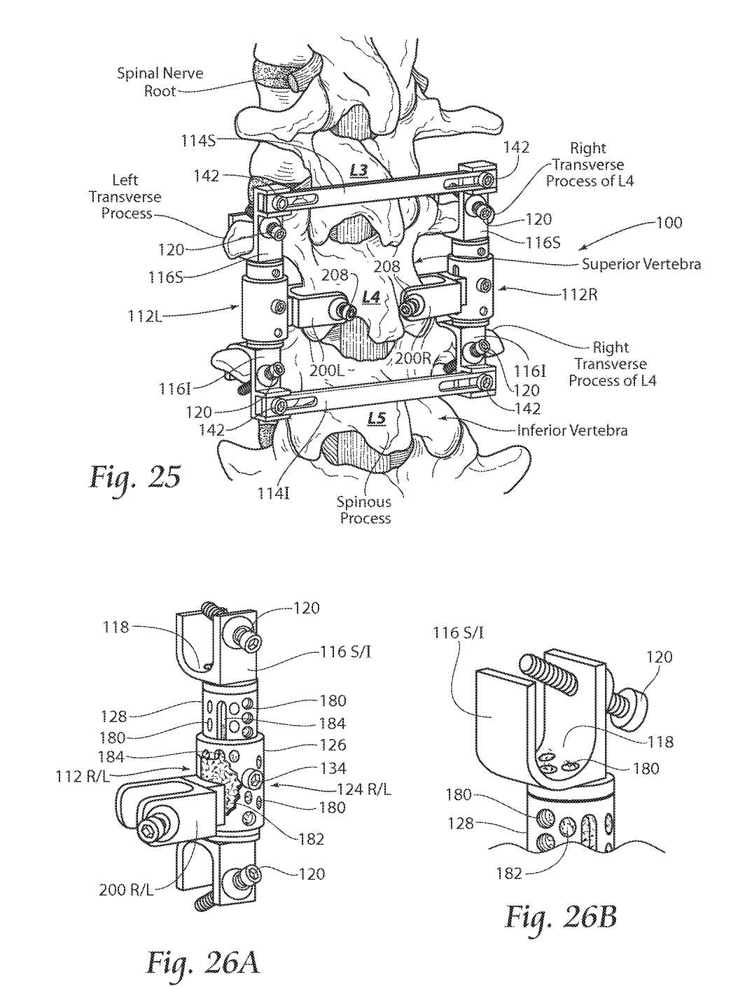

As FIG. 25 shows, the system 100, like the system 10, can further include a pair of transverse superior (or cranial) and inferior (or caudal) brace components, respectively 114S and 114I. Coupled by brace screws 142 to vertical extensions of the grip elements 116R and 116L, as previously described with respect to the system 10 (see FIG. 5), the superior and inferior brace components 114S and 114I are sized and configured, when assembled (as FIG. 25 shows), to couple superior and inferior regions of the lateral hoist assemblies 112R and 112L together. When properly assembled and installed (as FIG. 25 shows), the superior and inferior brace components 114S and 114I extending generally transversely across the longitudinal axis of the spine, between the lateral hoist assemblies. The superior and inferior brace components 114S and 114I of the system 100 exert a medial force upon the lateral hoist assemblies 112R and 112L (i.e., toward the spinous processes of the adjacent vertebrae), to further stabilize the lateral hoist assemblies 112R and 112L.

D. Posterolateral Fusion

The system 100 can also be adapted to achieve posterolateral spinal fusion between adjacent vertebrae posteriorly, without fusion within the disc space itself. As shown in FIGS. 26A and 26B, the cylinder component 126 and the rod component 128 of the support columns 124R and 124L can be modified to include an array of holes or fenestrations 180 and to accommodate inside the cylinder component 126 and rod component 128 the placement of bone graft, and fusion material 182. Desirably (see FIG. 26B), the inner curve of the u-shaped rests 118 also includes holes or fenestrations 180 that communicate with the interior of the adjoining cylinder component 126 or rod component 128, to allow the bone graft material 182 to extend to the transverse process.

### IV. CONCLUSION

The devices, systems, and methods that have been described exert posterolateral biomechanical force in the inter-transverse process space, combining both anterior and posterior distraction and stabilization, and achieving posterolateral fusion, optionally with facet fixation. The transverse process is located more anteriorly than the spinous process or even the facets. The transverse process is also closer to the discs in the anterior column of spine. Therefore, the net biomechanical effect of distracting the transverse processes is a combined anterior and posterior column distraction “indirectly”. The net biomechanical effect of distracting the transverse processes also avoids the potential for causing “kyphosis” or excessive forward bending/flexion angular deformity.

Other embodiments and uses of the inventions described herein will be apparent to those skilled in the art from consideration of the specification and practice of the inventions disclosed. The specification should be considered exemplary only. As will be easily understood by those of ordinary skill in the art, variations and modifications of each of the disclosed embodiments can be easily made within the scope of the invention.

I claim:

1. A system to be positioned between transverse processes of adjacent inferior and superior vertebrae in a spine for distraction and stabilization of an inter-transverse space in the spine and indirect expansion of both anterior and posterior spaces of the spine, the system comprising
at least one support component sized and configured to be mounted between selected left or right transverse processes of the adjacent vertebrae, the at least one support component exerting a separation force between the selected transverse processes generally longitudinally along the spine to distract and stabilize space between the adjacent inferior and superior vertebrae.

2. A system according to claim 1
wherein a length of the at least one support component is adjustable to vary the separation force.

3. A system according to claim 1 and further including
a fixation component coupled to the at least one support component, the fixation component being sized and configured to be fitted to a facet joint between the adjacent vertebrae to fixate the facet joint in tandem with the separation force.

4. A system to be positioned between left and right transverse processes of adjacent vertebrae in a spine to relieve pain associated with the spine by dynamic inter-transverse process distraction and stabilization and indirect expansion of both anterior and posterior spaces of the spine, the system comprising
a left support component sized and configured to be mounted between the left transverse processes of the adjacent vertebrae,
a right support component and configured to be mounted between the right transverse processes of the adjacent vertebrae, and
the left and right support components exerting a dynamic separation force between the left and right transverse processes generally longitudinally along the spine to distract and stabilize space between the adjacent inferior and superior vertebrae and indirectly expand both anterior and posterior spaces of the spine, thereby relieving pain.

5. A system to be positioned between left and right transverse processes of adjacent vertebrae in a spine to treat pain, numbness, and/or weakness of a leg by dynamic inter-transverse process distraction and stabilization and indirect expansion of both anterior and posterior spaces of the spine, the system comprising
a left support component sized and configured to be mounted between the left transverse processes of the adjacent vertebrae,
a right support component and configured to be mounted between the right transverse processes of the adjacent vertebrae, and
the left and right support components exerting a dynamic separation force between the left and right transverse processes generally longitudinally along the spine to distract and stabilize space between the adjacent inferior and superior vertebrae and indirectly expand both anterior and posterior spaces of the spine, thereby treat pain, numbness, and/or weakness of a leg.

6. A system to be positioned between left and right transverse processes of adjacent vertebrae in a spine for distraction and stabilization of an inter-transverse space in the spine and indirectly expansion of both anterior and posterior spaces of the spine, the system comprising
a left support component sized and configured to be mounted between the left transverse processes of the adjacent vertebrae,
a right support component and configured to be mounted between the right transverse processes of the adjacent vertebrae, and
the left and right support components exerting a separation force between the left and right transverse processes generally longitudinally along the spine to distract and stabilize space between the adjacent inferior and superior vertebrae and indirectly expand both anterior and posterior spaces of the spine.

7. A system according to claim 4 or 5 or 6
wherein a length of the at least one of the left and right support components is adjustable to vary the separation force.

8. A system according to claim 4 or 5 or 6
wherein a length of both the left and right support components is adjustable to vary the separation force.

9. A system according to claim 4 or 5 or 6 and further including
at least one brace component coupled to the left and right support components generally transversely across the spine, the at least one brace component being sized and configured to exert a medial stabilization force upon the left and right support components in tandem with the separation force.

10. A system according to claim 4 or 5 or 6 and further including
a first brace component coupled to an inferior region of both the left and right support components generally transversely across the spine,
a second brace component coupled to a superior region of both the left and right support components generally transversely across the spine,
the first and second brace components being sized and configured to exert medial stabilization forces upon the left and right support components in tandem with the separation force.

11. A system according to claim 4 or 5 or 6 and further including
a fixation component coupled to at least one of the left and right support components, the fixation component being sized and configured to be fitted to a facet joint between the adjacent vertebrae to fixate the facet joint in tandem with the separation force.

12. A system according to claim 11 and further including
at least one brace component coupled to the left and right support components generally transversely across the spine, the at least one brace component being sized and configured to exert a medial stabilization force upon the left and right support components in tandem with the separation force.

13. A system according to claim 4 or 5 or 6 and further including
a fixation component coupled to both the left and right support components, each fixation component being sized and configured to be fitted to a respective left and right facet joint between the adjacent vertebrae to mutually fixate the facet joints in tandem with the separation force.

14. A system according to claim 13 and further including
a first brace component coupled to an inferior region of both the left and right support components generally transversely across the spine,
a second brace component coupled to a superior region of both the left and right support components generally transversely across the spine,
the first and second brace components being sized and configured to exert medial stabilization forces upon the left and right support components in tandem with the separation force.

15. A system according to claim 4 or 5 or 6
wherein at least one of the left and right support components is sized and configured to carry a bone graft material.

16. A method for relieving pain associated with a spine comprising
accessing left or right transverse processes of adjacent vertebrae of a spine,
installing a support component in the inter-transverse process space between the accessed transverse processes, and
manipulating the support component to dynamically distract and stabilize the inter-transverse process space between the adjacent vertebrae and exert a separation force between the accessed transverse processes that indirectly expands both anterior and posterior spaces of the spine a sufficient amount to relieve the pain.

17. A method according to claim 16
wherein the pain is due to the development of spinal stenosis and the like.

18. A method according to claim 16
wherein the pain is due to the development of a disc herniation.

19. A method according to claim 16
wherein the pain comprises back pain.

20. A method according to claim 16
wherein installing the support component occurs without altering the accessed transverse processes.

21. A method according to claim 16 and further including
fusing at least one facet joint between the adjacent vertebrae in tandem with the separation force.

22. A method for relieving pain associated with a spine comprising
accessing left and right transverse processes of adjacent vertebrae of a spine,
installing left and right support components in the inter-transverse process space between the left and right transverse processes, and
manipulating at least one of the left and right support components to dynamically distract and stabilize the inter-transverse process space between the adjacent vertebrae and exert a separation force between the left and right transverse processes that indirectly expands both anterior and posterior spaces of the spine a sufficient amount to relieve the pain.

23. A method according to claim 22
wherein the pain is due to the development of spinal stenosis and the like.

24. A method according to claim 22
wherein the pain is due to the development of a disc herniation.

25. A method according to claim 22
wherein the pain comprises back pain.

26. A method according to claim 22
wherein installing the left and right support components occurs without altering the left and right transverse processes.

27. A method for treating pain, numbness, and/or weakness of a leg comprising
accessing left and right transverse processes of adjacent vertebrae of a spine,
installing left and right support components in the space between the left and right transverse processes, and
manipulating at least one of the left and right support components to dynamically distract and stabilize the inter-transverse process space between the adjacent vertebrae and exert a separation force between the left and right transverse processes that indirectly expands both anterior and posterior spaces of the spine a sufficient amount to relieve the pain, numbness, and/or weakness of the leg.

28. A method for stabilization of an intertransverse space in the spine comprising
accessing left and right transverse processes of adjacent vertebrae of a spine,
installing left and right support components in the space between the left and right transverse processes, and
manipulating at least one of the left and right support components to dynamically distract and stabilize the inter-transverse process space between the adjacent vertebrae and exert a separation force between the left and right transverse processes that indirectly expands both anterior and posterior spaces of the spine.

29. A method according to claim 22 or 27 or 28
wherein the manipulation includes adjusting a length of the at least one of the left and right support components to vary the separation force.

30. A method according to claim 22 or 27 or 28
wherein the manipulation includes adjusting a length of both the left and right support components to vary the separation force.

31. A method according to claim 22 or 27 or 28 and further including
exerting a medial stabilization force upon the left and right support component transversely across the spine in tandem with the separation force.

32. A method according to claim 31
wherein the medial stabilization force is exerted by coupling at least one brace component to the left and right support components transversely across the spine.

33. A method according to claim 22 or 27 or 28 and further including
fusing at least one facet joint between the adjacent vertebrae in tandem with the separation force.

34. A method according to claim 33
wherein the at least one facet joint is fused by fitting to the facet joint a fixation component that is coupled to at least one of the left and right support components.

35. A method according to claim 22 or 27 or 28 and further including
promoting fusion between the adjacent vertebrae by placing a bone graft material in the space between the left and right transverse processes.
